(19) United States
(12) Patent Application Publication
(10) Pub. No.: US 2014/0377316 A1
(43) Pub. Date: Dec. 25, 2014

(54) Home and personal care product and method for manufacturing the same

(71) Applicant: Kaffein Biotech Co., Ltd., Taoyuan City (TW)

(72) Inventors: Kai-Cheng Chuang, Taoyuan City (TW); Cheng-Hang Hsu, Taoyuan City (TW); Chih-Hsuan Wang, Taoyuan City (TW)

(21) Appl. No.: 14/315,290

(22) Filed: Jun. 25, 2014

(30) Foreign Application Priority Data
Jun. 25, 2013 (TW) 102122445
Jun. 13, 2014 (TW) 103120447

(51) Int. Cl. A61K 8/97; B02C 23/18; B02C 23/08; A61Q 19/10; A61Q 17/04; A61Q 11/00; A61Q 19/00; C11D 3/382; B01D 39/16; A61K 8/02

(52) U.S. Cl. CPC A61K 8/97; B01D 39/1646; B02C 23/18; B02C 23/08; A61K 8/0245; A61Q 17/04; A61Q 11/00; A61Q 19/00; C11D 3/382; A61Q 19/10; A61K 2800/41
USPC 424/401; 210/263; 241/24.11; 241/17; 241/19; 428/402; 424/59; 424/58; 424/725; 510/462; 510/344; 510/130; 510/137; 510/395

(57) ABSTRACT

The present invention discloses a method for manufacturing home and personal care product and a method for manufacturing degreased coffee particles thereof. The home and personal care product comprises coffee particles with different sizes, in which the coffee particles are degreased by physical and chemical methods and then grinded to different sizes to meet the requirements of different products. Natural compositions from original coffee can be further added for enhancing the clean and care effects of the product.

### NOTICE OF COPYRIGHT

A portion of the disclosure of this patent document contains material which is subject to copyright protection. The copyright owner has no objection to any reproduction by anyone of the patent disclosure, as it appears in the United States Patent and Trademark Office patent files or records, but otherwise reserves all copyright rights whatsoever.

### CROSS REFERENCE OF RELATED APPLICATION

This Non-provisional application claims priority under 35 U.S.C. §119(a) on Patent Application Nos. 102122445 and 103120447 filed in Taiwan, the entire contents of which are hereby incorporated by reference.

### BACKGROUND OF THE PRESENT INVENTION

1. Field of Invention

This invention relates to a home and personal care product and a method for manufacturing the same and, more particularly, to a home and personal care product comprising coffee particles and a method for manufacturing the same.

2. Description of Related Arts

Home and personal care product comprises such as soap, laundry detergent, body wash, facial cleaner, scrub, exfoliator, exfoliating clay, toothpaste, filtering and purifying product (such as air filter and water purifier), anti-UV spray, facial mask, shampoo, hair conditioner, hair styling product, shower gel, fabric washing powder and liquid, fabric softener, laundry dryer aids, easily-iron spray, deodorant stick, wax and polish (such as wax for car and furniture), cleaning spray, abrasive cleaner, carpet cleaner, cosmetic, lipstick, eye shadow, skin cream, massage oil, lotion, ointment, suntan lotion, shaving product, depilatory product, hair colorant, paint, varnish and lacquer (such as brickwork sealant), and so on. The current various home and personal care products require multi-functions. Taking early cleaning products as an example, they only can be utilized to remove oil or dirty. However, as technology progressing, many researchers make efforts in finding additional functions for the cleaning product. For example, the cleaning product can also have a conditioning effect.

Caffeine and coffee polyphenol of coffee allow skin to have an anti-oxidizing effect, maintain the humidity of the skin, lubricate the skin, allow the skin to be stretched taut and prevent the skin from chapping. They also can stimulate the skin to decompose fat. In particular, some portions, which hoard fat easily, such as thigh, belly, arm, and hip, can use personal care product containing coffee for not only cleaning and caring but also massaging and eliminating fat. Because coffee has a plurality of compositions and specific functions, it can also be applied on home care products. And further, coffee is a natural substance so that it will not pollute the environment. To sum up, the method for applying coffee on the home and personal care product is extremely required.

### SUMMARY OF THE PRESENT INVENTION

A home or personal care product according to an embodiment of the present invention comprises a plurality of degreased coffee particles having a size between 50 nm and 500 μm.

A method for manufacturing home or personal care product according to an embodiment of the present invention comprises a step of degreasing, drying and grinding a plurality of coffee particles to allow the coffee particles to have a size between 50 nm and 500 μm, and a step of taking the coffee particles to manufacture a home or personal care product.

### BRIEF DESCRIPTION OF THE DRAWINGS

FIG. 1 is a flowchart showing a method for manufacturing coffee particles according to an embodiment of the present invention;

FIG. 2 is a flowchart showing a method for manufacturing a soap comprising coffee particles according to an embodiment of the present invention;

FIG. 3 is a flowchart showing a method for manufacturing a liquid home or personal care product comprising coffee particles according to an embodiment of the present invention; and

FIG. 4 is a flowchart showing a method for manufacturing a home or personal care product comprising coffee particles according to an embodiment of the present invention.

### DETAILED DESCRIPTION OF THE PREFERRED EMBODIMENT

Specific embodiments of the present invention are further described in the following and cooperated with the attached figures for allowing the abovementioned, other aspects, features and advantages of the present invention to be easily understood.

The present invention provides a home or personal care product and a method for manufacturing thereof. The home or personal care product comprises degreased coffee particles having a size between 50 nm and 500 μm. If the size of the coffee particles is too large, users may be scratched. If the size of the coffee particles is too small, they will easily aggregate due to molecular force and cannot be efficiently dispersed into the home or personal care product.

In an embodiment of the present invention, the coffee particles can be divided into a plurality of coarse coffee particles having a size between 300 μm and 500 μm and a plurality of fine coffee particles having a size between 50 nm and 250 μm for improving the cleaning effect. In an embodiment of the present invention, a weight ratio of the coarse coffee particles and the fine coffee particles is ranged from 1:10000 to 1:5. If the content of the coarse coffee particles is too high, the product is similar to a product without the fine coffee particles. If the content of the coarse coffee particles is too low, the product is similar to a product without the coarse coffee particles.

The steps for degreasing the coffee particles are described as the following. In an embodiment of the present invention, the source of the coffee particles can be roasted recycled coffee grounds or ground green beans. It is noted that the term “degrease” used in the specification does not mean the removal of only the grease of the coffee particles but also other non-polar substances. And further, partial or whole non-polar substance can be removed.

In an embodiment of the present invention, supercritical CO2, which has a pressure between 8 MPa and 45 MPa and a temperature between 35° C. and 70° C., can be utilized to deal with preliminary ground coffee particles for degreasing the coffee particles.

In an embodiment of the present invention, the preliminary ground coffee particles are placed into water and heated to 50˜100° C. to form a oily matter on surface of water and then the oily matter is removed. The coffee particles are second washed by water and sonicated for removing tannins and pigment. After filtering water, the wet coffee particles will be baked for removing water and drying. In an embodiment of the present invention, a dry grinder can be utilized to grind and fine the abovementioned degreased coffee particles and filter out different sizes of coffee particles. The coffee particles, which have been grinded and fined by the dry grinder, have a micro-scaled size, such as between 1 μm and 500 μm. However, the present invention is not limited thereto.

In an embodiment of the present invention, a wet grinder, such as a nano bead mill, a grinder or a kneading machine, can be utilized to miniaturize the micro-scaled coffee particles if the nano-scaled coffee particles are required. In wet grinding, quaternary-ammonium-group-containing cationization agent can be added so that the chemical modified coffee particles carry positive charges so as to disperse easily. However, the present invention is not limited thereto. In addition to the abovementioned chemical modification, the coffee particles can also be passed through a small hole with a high-voltage positive charge to carry positive charges. However, the present invention is not limited thereto. The purpose of such the physical modification is also to disperse the coffee particles easily.

It is noted that the coffee particles are difficult to be grinded in the following miniaturizing process if the coffee particles does not been degreased. And further, the mesh screen will be easily blocked up when the coffee particles are classified according to size. The grease will further allow the coffee particles of the home or personal care product to aggregate easily. In addition, non-degreased coffee particles deteriorate easily and mildew so that the lifetime of the home or personal care product is decreased. On the other hand, the grease of the coffee particles will consume the content of base and increase the difficulty of the manufacture if the coffee particles are utilized for manufacturing the soap. The base can be sodium hydroxide, however, the present invention is not limited thereto. For example, the amount of the grease in each lot of the coffee particles is different so that the amount of the grease in the coffee particles needs to be further quantified before the manufacture of the soap to avoid the insufficiency of sodium hydroxide.

And then, the degreased, dried, grinded and classified coffee particles will be taken to manufacturing home or personal care products, such as soap, laundry detergent, body wash, facial cleaner, scrub, exfoliator, exfoliating clay, toothpaste, filtering and purifying product (such as air filter or water purifier), anti-UV spray, facial mask or other similar products.

In an embodiment of the present invention, the home or personal care product is a soap having a soap hardness between 130 and 155. The soap comprises 0.1˜25 weight % of the coffee particles and the coffee particles have a size between 1 μm and 500 μm. If the hardness of the soap is too high, it is difficult to form foam so that the cleaning ability is low. If the hardness of the soap is too low, it is easier to be softened so that it will be consumed too fast. If the content of the coffee particles is too high, it is difficult to form foam so that the cleaning effect is low. If the content of the coffee particles is too low, the exfoliating effect is decreased. If the size of the coffee particles is too large, the skin of the user may be scratched. If the size of the coffee particles is too small, the exfoliating effect is decreased and there is no granular sensation.

In an embodiment of the present invention, the home or personal care product is a laundry detergent comprising 0.01˜15 weight % of the coffee particles. The coffee particles have a size between 50 nm and 300 nm. After the clothes is washed, the coffee particles will remain in the fabric of the clothes for achieving an anti-UV effect. To allow the coffee particles to easily attach to the fabric of the clothes, the coffee particles can be further modified to carry charges. If the content of the coffee particles is too high, the cleaning effect is low. If the content of the coffee particles is too low, it has no anti-UV effect. If the size of the coffee particles is too large, they cannot be remained in the fabric of the clothes. If the size of the coffee particles is too small, UV rays cannot be efficiently isolated.

In an embodiment of the present invention, the home or personal care product is a body wash comprising 0.1˜30 weight % of the coffee particles and the coffee particles have a size between 50 μm and 500 μm. If the content of the coffee particles is too high, the cleaning effect will be low. If the content of the coffee particles is too low, the exfoliating effect will be low. If the size of the coffee particles is too large, the skin may be scratched. If the size of the coffee particles is too small, the exfoliating effect is low and there is no granular sensation.

In an embodiment of the present invention, the home or personal care product is a facial cleaner comprising 0.1˜10 weight % of the coffee particles and the coffee particles have a size between 1 μm and 400 μm. If the content of the coffee particles is too high, the cleaning effect will be low. If the content of the coffee particles is too low, the exfoliating effect will be low. If the size of the coffee particles is too large, the skin may be scratched. If he size of the coffee particles is too small, the exfoliating effect is low and there is no granular sensation.

In an embodiment of the present invention, the home or personal care product is a sunscreen comprising 0.01˜10 weight % of the coffee particles and the coffee particles have a size between 50 nm and 1 μm. If the content of the coffee particles is too high, it is not beautiful. If the content of the coffee particles is too low, UV rays cannot be efficiently isolated. If the size of the coffee particles is too large, it is not beautiful. If the size of the coffee particles is too small, UV rays cannot be efficiently isolated. The coffee particles are mainly used to replace titanium dioxide or zinc oxide existed in the compositions of the sunscreen for achieving natural and physical sunscreen.

In an embodiment of the present invention, the home or personal care product is a scrub comprising 5˜50 weight % of the coffee particles and the coffee particles have a size between 100 μm and 500 μm. If the content of the coffee particles is too high, the skin may be scratched. If the content of the coffee particles is too low, the exfoliating effect will be low and there is no granular sensation. If the size of the coffee particles is too large, the skin may be scratched. If the size of the coffee particles is too small, the exfoliating effect is low and there is no granular sensation.

In an embodiment of the present invention, the home or personal care product is a toothpaste comprising 0.1˜30 weight % of the coffee particles and the coffee particles have a size between 1 μm and 200 μm. If the content of the coffee particles is too high, the tooth enamel may be damaged. If the content of the coffee particles is too low, the deep cleaning effect is decreased. If the size of the coffee particles is too large, the tooth enamel may be damaged. If the size of the coffee particles is too small, the deep cleaning effect is decreased.

In an embodiment of the present invention, the home or personal care product is a filtering and purifying material, such as air filter or water purifier, comprising 5˜50 weight % of the coffee particles and the coffee particles have a size between 100 nm and 500 μm. If the content of the coffee particles is too high, they cannot be formed with other materials easily. If the content of the coffee particles is too low, there is no deodorizing effect. If the size of the coffee particles is too large, they cannot be formed with other materials easily. If the size of the coffee particles is too small, they will be dissolved in the filtered object easily.

In an embodiment of the present invention, the home or personal care product is an anti-UV spray comprising 0.0110 ˜weight % of the coffee particles and the coffee particles have a size between 50 nm and 1 μm. If the content of the coffee particles is too high, it cannot be sprayed out easily. If the content of the coffee particles is too low, UV rays cannot be efficiently isolated. If the size of the coffee particles is too large, it cannot be sprayed out easily. If the size of the coffee particles is too small, UV rays cannot be efficiently isolated. The anti-UV spray is applied on the clothes to allow the clothes to have an anti-UV effect or on the skin to allow the skin to have an anti-UV effect.

In an embodiment of the present invention, the home or personal care product is a facial mask comprising 0.1˜10 weight % of the coffee particles and the coffee particles have a size between 1 μm and 500 μm. If the content of the coffee particles is too high, the skin may be scratched. If the content of the coffee particles is too low, the exfoliating effect will be low and there is no granular sensation. If the size of the coffee particles is too large, the skin may be scratched. If the size of the coffee particles is too small, the exfoliating effect is low and there is no granular sensation.

In the method for manufacturing the home or personal care product comprising coffee particles according to an embodiment of the present invention, the manufacture of raw material of the home or personal care product is performed first. If the finished product of the home or personal care product is liquid, degreased coffee particles with an appropriate ratio and size are added after finishing the liquid raw material and stirred. If the finished product of the home or personal care product is solid, degreased coffee particles with an appropriate ratio and size are added when the raw material of the home or personal care product is under the thick state and then stirred continuously for evenly mixing. And then, a step of solidifying is performed.

In an embodiment of the present invention, a physical or chemical treatment can be further applied on the solution during the water-washing procedure to collect beneficial chemical components for the skin of the human body, such as caffeic acid, caffeic grease, caffeine, trigonellinelline, chlorogenic acid, vitamin B and so on, through refining, extraction and separation, respectively. And then, the beneficial chemical components can be further added during the manufacture of the home or personal care product for achieving individual specific purposes.

In an embodiment of the present invention, caffeine can further added when the home or personal care product is a facial cleaner or body wash. The low dose caffeine can be percutaneously absorbable for improving the metabolic function of the human body and achieving a freshening effect.

In an embodiment of the present invention, the component having natural acid substance, such as caffeic acid, can be added when the home or personal care product is an exfoliating product, such as a scrub, for softening stratum corneum of the human body and improving the efficiency of the exfoliating product.

In an embodiment of the present invention, chlorogenic acid and trigonellinelline from natural coffee beans can be added into the home or personal care product for moisturizing and relieving the skin (trigonellinelline), for anti-oxidation (chlorogenic acid) and for antimicrobial effect (chlorogenic acid and trigonellinelline).

To allow the abovementioned, other aspects, features and advantages of the present invention to be easily understood, several embodiments of the present invention are further described and cooperated with the attached figures as the following:

### Embodiment 1 (Preparation of Degreased Coffee Particles)

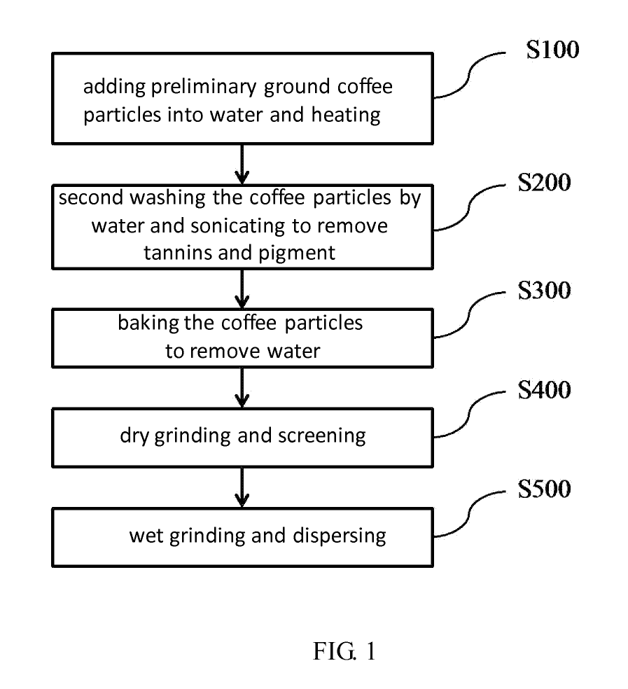

FIG. 1 is a flowchart showing a method for manufacturing coffee particles according to an embodiment of the present invention. In step S100, the preliminary ground coffee particles are added into water and heated to 50˜100° C., in which a weight ratio of water and the coffee particles is around 5:1. An oily matter, which floats on surface of water, (non-polar substance) is then removed. In step S200, the coffee particles are second washed by water and are sonicated to remove tannins and pigment. In step S300, water is filtered and the moist coffee particles is baked at a temperature lower than 70° C. for drying the coffee particles. In step S400, the dry grinder is utilized to grind and fine the coffee particles. The coffee particles having different sizes are then filtered out.

A physical or chemical treatment can be further applied on the oily matter or water solution, which is collected in the above water washing procedure, to collect beneficial chemical components for the skin of the human body, such as caffeic acid, caffeic grease, caffeine, trigonellinelline, chlorogenic acid, vitamin B and so on, through refining, extraction and separation, respectively. And then, the beneficial chemical components can be further added during the manufacture of the cleaning product for achieving individual specific purposes. Chlorogenic acid and trigonellinelline contained in the coffee beans can be utilized for moisturizing and relieving the skin (trigonellinelline), for anti-oxidation (chlorogenic acid) and for antimicrobial effect (chlorogenic acid and trigonellinelline). Coffee can also be utilized to prevent skin cancer.

The coffee particles, which are filtered out in step S400, have a size between tens of μm and 500 μm. If nano-scaled coffee particles are required, a step S500 is performed. A wet grinder, such as a nano bead mill, a grinder or a kneading machine, can be utilized to miniaturize the micro-scaled coffee particles. In wet grinding, quaternary-ammonium-group-containing cationization agent can be added so that the chemical modified coffee particles carry positive charges so as to disperse easily. A physical modification is another method to allow the coffee particles to carry positive charges. In details, the coffee particles will be passed through a small hole with a high-voltage positive charge to carry positive charges. Basically, the steps for manufacturing coffee particles can be simplified to steps of removing water, grinding and then filtering different sizes of coffee particles.

### Embodiment 2 (Preparation of Soap Comprising Coffee Particles)

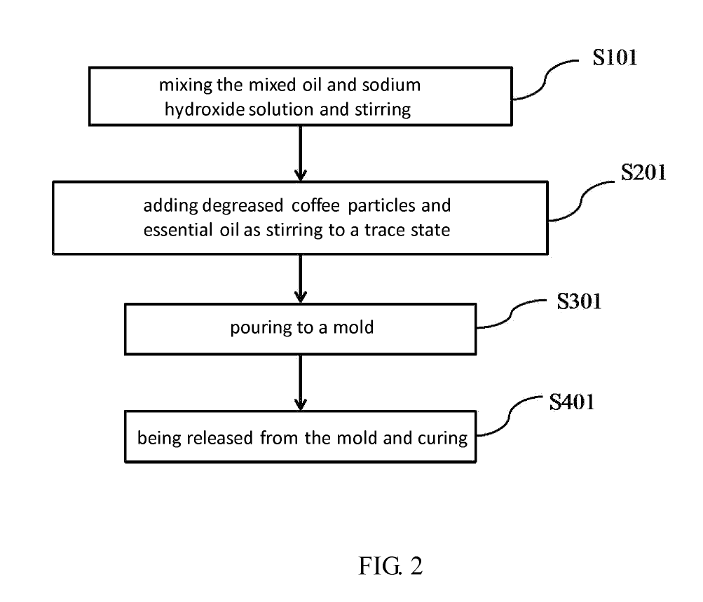

FIG. 2 is a flowchart showing a method for manufacturing a soap comprising coffee particles according to an embodiment of the present invention. In step S101, 200 g of olive oil, 200 g of palm oil and 150 g of coconut oil are mixed to form a mixed oil and heated to 50° C. 87 g of sodium hydroxide is added into 210 g of water. The sodium hydroxide solution is added into the mixed oil and stirred after cooling to around 50° C. In step S201, the abovementioned mixture is stirred continuously to a trace state. 20 g of degreased coffee particles having a size between 200 μm and 300 μm and essential oil are added into the abovementioned mixture and are stirred uniformly. It supposes that the abovementioned step needs 40 minutes and thus the coffee particles and the essential oil can be added between 30 minutes and 40 minutes. In step S301, the solution obtained in step S201 is poured into a mold. In step S401, the manufacture of the soap comprising coffee particles is accomplished after the soap is released from the mold and placed under a cool and ventilated place for 4 to 5 weeks for curing.

Different kinds of oils can be utilized during the manufacture of the soap for controlling the hardness of the soap (INS). Through the experiment, the product will has a better feeling when the soap hardness is ranged from 130 to 155 and the size of the coffee particles is ranged from 1 to 500 μm, most preferably from 50 to 500 μm. A weight ratio of the added coffee particles and the soap is ranged from 0.1 to 25 weight %, preferably from 0.1 to 10 weight %, most preferably from 0.5 to 3.5 weight %. That is, the soap contains 0.1˜25 weight % of the coffee particles, preferably 0.1˜10 weight %, most preferably 0.5˜3.5 weight %.

### Embodiment 3 (Preparation of Liquid Home or Personal Care Product)

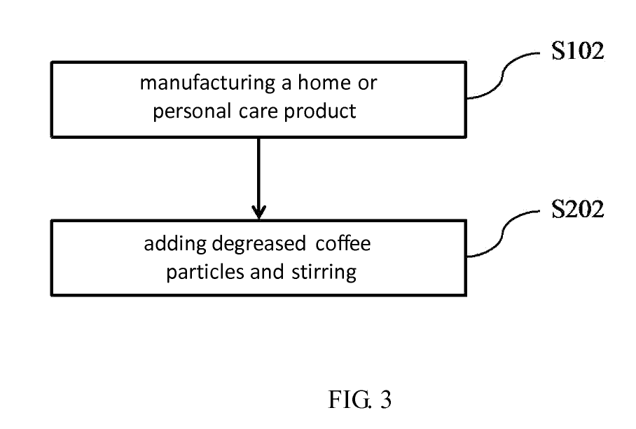

FIG. 3 is a flowchart showing a method for manufacturing a liquid home or personal care product comprising coffee particles according to an embodiment of the present invention. In step S102, the fundamental compositions of the liquid home or personal care product, such as laundry detergent, body wash, scrub, sunscreen and facial cleaner are manufactured. In step S202, the degreased coffee particles are added into the liquid home or personal care product and continuously stirred for evenly mixing.

### Embodiment 4 (Preparation of Home or Personal Care Product)

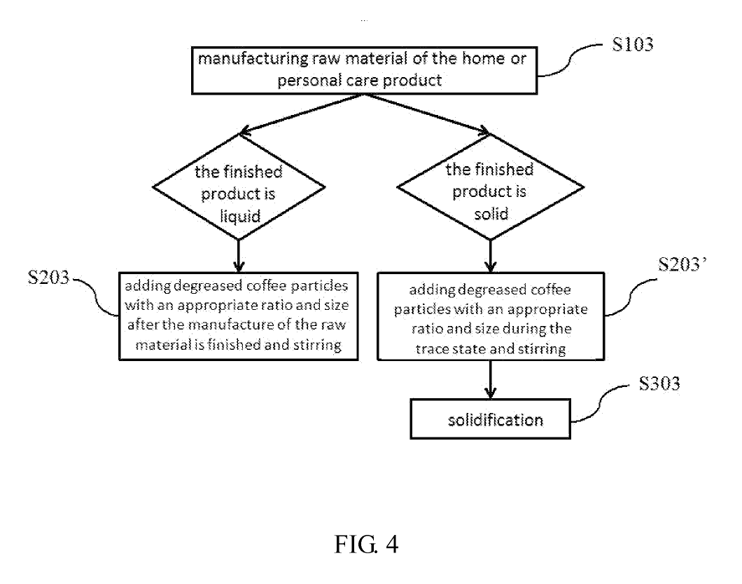

FIG. 4 is a flowchart showing a method for manufacturing a home or personal care product comprising coffee particles according to an embodiment of the present invention. In step S103, the raw material of the home or personal care product is manufactured first. If the finished product of the home or personal care product is liquid, a step S203 is performed to add degreased coffee particles with an appropriate ratio and size after the manufacture of the raw material of the home or personal care product is finished and to stir. If the finished product of the home or personal care product is solid, a step S203′ is performed to add degreased coffee particles with an appropriate ratio and size when the raw material of the home or personal care product is under the trace state and then to continuously stir for evenly mixing. And then, a step S303 of such as solidifying is performed. When the coffee particles are added into the raw material of the home or personal care product, hot air and negative ions can be applied on the coffee particles for improving the uniformity of the coffee particles in the home or personal care product.

### Embodiment 5 (Body Exfoliator)

Table 1 shows composition of the body exfoliator. It utilizes degreased coffee particles, in which the body exfoliator comprises 5˜50 weight % of the coffee particles, preferably 10˜30 weight %, and the coffee particles have a size between 100 and 500 μm, preferably between 200 and 400 μm. The method for manufacturing thereof can refer to FIG. 3 and FIG. 4.

TABLE 1
Compositions of body exfoliator

coffee particles
Water
Mineral Oil
Caprylic Triglyceride
Ceterayl Olivate, Sorbitan Olivate
Butyrospermum Parkii Butter
Avocado Oil
Urea
Beeswax
Panthenol
Olea Europaea (Olive) Oil
Saccharide Isomerate
Xanthum Gum
Ceramide3; Ceramide6 II; Ceramide1; Phytosphingosine;
Cholesterol; Sodium Lauroyl Lactylate; Carbomer; Xanthan
Gum
Fragrance
Methyl Paraben
Phenoxyehanol

### Embodiment 6 (Skin Cleaner)

Table 2 shows composition of the skin cleaner. It utilizes degreased coffee particles, in which the skin cleaner comprises 0.1˜30 weight % of the coffee particles, preferably 0.5˜10 weight %, and the coffee particles have a size between 50 and 500 μm, preferably between 100 and 400 μm. The method for manufacturing thereof can refer to FIG. 3 and FIG. 4.

TABLE 2
Compositions of skin cleaner

coffee particles
Water
Sodium Lauryl Ether Sulfate
Sodium Cocoyl Isethionate
Cocamidopropyl Betaine
Soyamide DEA
PEG-7 Glyceryl Cocoate
Saccharideisomerate
Cucumis Sativus (Cucumber) Extract
D-Panthenol
Sodium Chloride
Cetyl Alcohol
Fragrance
Methyl Paraben
Methylisothiazolinone

### Embodiment 7 (Amino Acid Cleaning Cream)

Table 3 shows composition of the amino acid cleaning cream. It utilizes degreased coffee particles, in which the amino acid cleaning cream comprises 0.1˜10 weight % of the coffee particles, preferably 0.5˜5 weight %, and the coffee particles have a size between 1 and 400 μm, preferably between 150 and 300 μm. The method for manufacturing thereof can refer to FIG. 3 and FIG. 4.

TABLE 3
Compositions of amino acid cleaning cream

coffee particles
Sodium Cocoyl Isethionate
Sodium Methyl Cocoyl Taurate
TEA-Lauroyl Glutamate
PEG-30 Glyceryl Cocoate
Sodium Hyaluronate
Dipotassium Glycyrrhetinate

### Embodiment 8 (Exfoliating Clay)

Table 4 shows composition of the exfoliating clay. It utilizes degreased coffee particles, in which the exfoliating clay comprises 0.1˜30 weight % of the coffee particles, preferably 1˜20 weight %, and the coffee particles have a size between 50 and 400 μm, preferably between 150 and 300 μm. The method for manufacturing thereof can refer to FIG. 3 and FIG. 4.

TABLE 4
Compositions of exfoliating clay

coffee particles
Steareth-21
Steareth-2
Stearic Acid
Isohexadecane
α-bisabolol
1.3-Butylene Glycol
Aluminum Silicate
Water
Caffeine & Cyclodextrin
Laminaria Saccharina Extract
Saccharideisomerate
Arbutin
Hydrolyzed Royal Jelly Protein
White Willow Bark Extract
Butylene Glycol, Vibrio Alginolyticus Ferment Filtrate
Algae Extract
Glycerine
Polyethylene
Fragrance
Phenoxyethanol
Methylisothiazolinone

Although the present invention has been described in considerable detail with reference to certain preferred embodiments thereof, the disclosure is not for limiting the scope of the invention. Persons having ordinary skill in the art may make various modifications and changes without departing from the scope and spirit of the invention. Therefore, the scope of the appended claims should not be limited to the description of the preferred embodiments described above.

What is claimed is:

1. A home or personal care product, comprising:
a plurality of degreased coffee particles having a size between 50 nm and 500 μm.

2. The home or personal care product according to claim 1, wherein the coffee particles are divided into a plurality of coarse coffee particles having a size between 300 μm and 500 μm and a plurality of fine coffee particles having a size between 50 nm and 250 μm and a weight ratio of the coarse coffee particles and the fine coffee particles is ranged from 1:10000 to 1:5.

3. The home or personal care product according to claim 1, wherein the surface of the coffee particles is electrically charged after a chemical or physical modification.

4. The home or personal care product according to claim 1 is a soap having a soap hardness between 130 and 155, wherein the soap comprises 0.1˜25 weight % of the coffee particles and the coffee particles have a size between 1 μm and 500 μm.

5. The home or personal care product according to claim 1 is a laundry detergent comprising 0.01˜15 weight % of the coffee particles, wherein the coffee particles have a size between 50 nm and 300 nm.

6. The home or personal care product according to claim 1 is a body wash comprising 0.1˜30 weight % of the coffee particles, wherein the coffee particles have a size between 50 μm and 500 μm.

7. The home or personal care product according to claim 1 is a facial cleaner comprising 0.1˜10 weight % of the coffee particles, wherein the coffee particles have a size between 1 μm and 400 μm.

8. The home or personal care product according to claim 1 is a sunscreen comprising 0.01˜10 weight % of the coffee particles, wherein the coffee particles have a size between 50 nm and 1 μm.

9. The home or personal care product according to claim 1 is a scrub comprising 5˜50 weight % of the coffee particles, wherein the coffee particles have a size between 100 μm and 500 μm.

10. The home or personal care product according to claim 1 is a tooth paste comprising 0.1˜30 weight % of the coffee particles, wherein the coffee particles have a size between 1 μm and 200 μm.

11. The home or personal care product according to claim 1 is a filtering and purifying material comprising 5˜50 weight % of the coffee particles, wherein the coffee particles have a size between 100 nm and 500 μm.

12. The home or personal care product according to claim 1 is an anti-UV spray comprising 0.01˜10 weight % of the coffee particles, wherein the coffee particles have a size between 50 nm and 1 μm.

13. The home or personal care product according to claim 1 is a facial mask comprising 0.1˜10 weight % of the coffee particles, wherein the coffee particles have a size between 1 μm and 500 μm.

14. A method for manufacturing home or personal care product, comprising:
degreasing, drying and grinding a plurality of coffee particles to allow the coffee particles to have a size between 50 nm and 500 μm; and
taking the coffee particles to manufacture a home or personal care product.

15. The method for manufacturing home or personal care product according to claim 14, wherein the step of degreasing the coffee particles comprises:
placing the coffee particles into water, heating them to 50˜100° C. to form an oily matter on surface of water and removing the oily matter.

16. The method for manufacturing home or personal care product according to claim 14, wherein the step of degreasing the coffee particles comprises:
degreasing the coffee particles using supercritical CO₂, wherein the supercritical CO₂ has a pressure between 8 MPa and 45 MPa and has a temperature between 35° C. and 70° C.
